(19) United States
(12) Patent Application Publication
(10) Pub. No.: US 2026/0022107 A1
(43) Pub. Date: Jan. 22, 2026

(54) AMINOMETHYL CARBAMATE COMPOUNDS AND DERIVATIVES AND METHODS OF THEIR MAKING

(71) Applicant: CURIA GLOBAL, INC., Albany, NY (US)

(72) Inventors: Pulla Reddy SINGAM, Northbrook, IL (US); Vijaykumar PAIKE, Aurangabad (IN); Bhaskar KUMMARI, Kukatpally Hyderabad (IN); Dinesh KALBHOR, Aurangabad (IN)

(21) Appl. No.: 19/269,322

(22) Filed: Jul. 15, 2025

Related U.S. Application Data
(60) Provisional application No. 63/671,923, filed on Jul. 16, 2024.

(51) Int. Cl. C07D 401/12; C07D 211/14; C07D 213/74

(52) U.S. Cl. CPC C07D 401/12; C07D 211/14; C07D 213/74

(57) ABSTRACT

The present disclosure is directed to a compound of Formula (I) and a process for preparation of a compound of Formula (I):

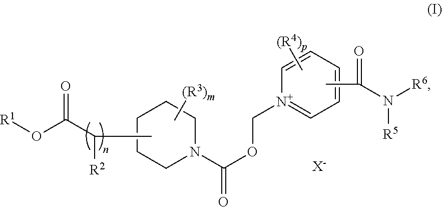

wherein R¹, R², R³, R⁴, R⁵, R⁶, X, n, m, and p are defined as set forth herein. Also disclosed is a compound of Formula (II):

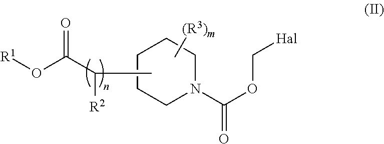

wherein Hal, R¹, R², R³, and n are defined as set forth herein.

This application claims the priority benefit of U.S. Provisional Patent Application Ser. No. 63/671,923, filed Jul. 16, 2024, which is hereby incorporated by reference in its entirety.

### FIELD

The present application relates to aminomethyl carbamate compounds and derivatives thereof, as well as methods for making the same.

### BACKGROUND

Aminomethyl carbamate compounds are generally prepared by treating an amine with chloromethyl chloroformate and the resulting chloromethyl derivative is coupled with another amine. This process does not work well when a free carboxyl group is present as the carboxyl group reacts with chloromethyl chloroformate. So, first the carboxyl group needs to be protected before the amine can be reacted with chloromethyl chloroformate.

The present disclosure is directed to overcoming these and other deficiencies in the art.

### SUMMARY

One aspect of the present disclosure relates to a process for preparation of a compound of Formula (I):

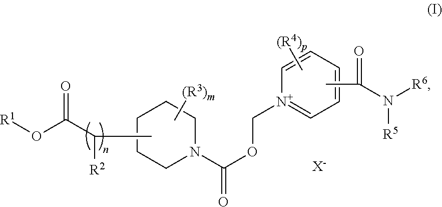

wherein
   R¹ is C₁-C₆ alkyl;
   R² is independently selected from the group consisting of H, C₁-C₆ alkyl, C₃-C₇ cycloalkyl, C₄-C₁₃ cycloalkylalkyl, heterocyclyl, aryl, and heteroaryl, wherein C₁-C₆ alkyl, C₃-C₇ cycloalkyl, C₄-C₁₃ cycloalkylalkyl, heterocyclyl, aryl, and heteroaryl can be optionally substituted from 1 to 3 times with a substituent selected independently at each occurrence from the group consisting of C₁-C₆ alkyl, OH, halogen, and C₁-C₆ alkoxy;
   R³ is independently selected at each occurrence thereof from the group consisting of H, C₁-C₆ alkyl, C₃-C₇ cycloalkyl, C₄-C₁₃ Cycloalkylalkyl, heterocyclyl, aryl, and heteroaryl;
   R⁴ is independently selected at each occurrence thereof from the group consisting of H, C₁-C₆ alkyl, C₃-C₇ cycloalkyl, C₄-C₁₃ cycloalkylalkyl, heterocyclyl, aryl, and heteroaryl;
   R⁵ is selected from the group consisting of H, C₁-C₆ alkyl, C₃-C₇ cycloalkyl, C₄- C₁₃ cycloalkylalkyl, heterocyclyl, aryl, and heteroaryl, wherein C₁-C₆ alkyl, C₃-C₇ cycloalkyl, C₄-C₁₃ cycloalkylalkyl, heterocyclyl, aryl, and heteroaryl can be optionally substituted from 1 to 3 times with a substituent selected independently at each occurrence from the group consisting of —OC₁-C₆ alkyl, —C(O)OH, and —C(O)OC₁-C₆ alkyl;
   R⁶ is selected from the group consisting of H, C₁-C₆ alkyl, C₃-C₇ cycloalkyl, C₄- C₁₃ cycloalkylalkyl, heterocyclyl, aryl, and heteroaryl, wherein C₁-C₆ alkyl, C₃-C₇ cycloalkyl, C₄-C₁₃ Cycloalkylalkyl, heterocyclyl, aryl, and heteroaryl can be optionally substituted from 1 to 3 times with a substituent selected independently at each occurrence from the group consisting of —OC₁-C₆ alkyl, —C(O)OH, and —C(O)OC₁-C₆ alkyl;
   X is halogen, mesylate, tosylate, or triflate;
   n is 0, 1, 2, 3, 4, 5, or 6;
   m is 0, 1, 2, 3, 4, 5, 6, 7, 8, or 9; and
   p is 0, 1, 2, 3, or 4;
      or a solvate thereof. This process includes:
providing an intermediate compound of Formula (II) having the structure:

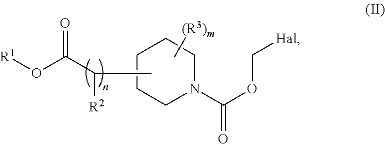

wherein Hal is halogen; and
forming the compound of Formula (I) from the intermediate compound of Formula (II).

In a preferred embodiment of the above process, the step of providing an intermediate compound of Formula (II) comprises providing an intermediate compound of Formula (IVa) and/or Formula (IVb):

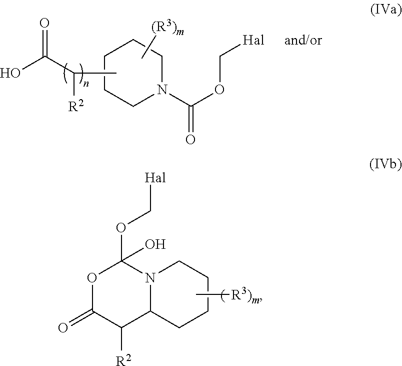

wherein Hal is halogen; and
reacting the intermediate compound of Formula (IVa) and/or Formula (IVb) with an alkylating agent to produce the intermediate compound of Formula (II).

In a preferred embodiment of the above process, the intermediate compound of Formula (II) is reacted with a compound of Formula (III):

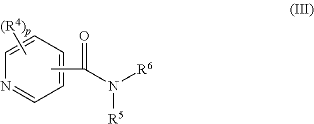

to produce the compound of Formula (I).

Another aspect of the present disclosure relates to a compound of Formula (I):

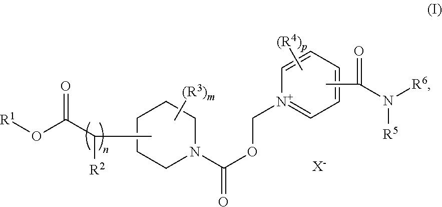

wherein
R¹ is C₁-C₆ alkyl;
R² is independently selected from the group consisting of H, C₁-C₆ alkyl, C₃-C₇ cycloalkyl, C₄-C₁₃ cycloalkylalkyl, heterocyclyl, aryl, and heteroaryl, wherein C₁-C₆ alkyl, C₃-C₇ cycloalkyl, C₄-C₁₃ cycloalkylalkyl, heterocyclyl, aryl, and heteroaryl can be optionally substituted from 1 to 3 times with a substituent selected independently at each occurrence from the group consisting of C₁-C₆ alkyl, OH, halogen, and C₁-C₆ alkoxy;
R³ is independently selected at each occurrence thereof from the group consisting of H, C₁-C₆ alkyl, C₃-C₇ cycloalkyl, C₄-C₁₃ cycloalkylalkyl, heterocyclyl, aryl, and heteroaryl;
R⁴ is independently selected at each occurrence thereof from the group consisting of H, C₁-C₆ alkyl, C₃-C₇ cycloalkyl, C₄-C₁₃ cycloalkylalkyl, heterocyclyl, aryl, and heteroaryl;
R⁵ is selected from the group consisting of H, C₁-C₆ alkyl, C₃-C₇ cycloalkyl, C₄-C₁₃ cycloalkylalkyl, heterocyclyl, aryl, and heteroaryl, wherein C₁-C₆ alkyl, C₃-C₇ cycloalkyl, C₄-C₁₃ cycloalkylalkyl, heterocyclyl, aryl, and heteroaryl can be optionally substituted from 1 to 3 times with a substituent selected independently at each occurrence from the group consisting of —OC₁-C₆ alkyl, —C(O)OH, and —C(O)OC₁-C₆ alkyl;
R⁶ is selected from the group consisting of H, C₁-C₆ alkyl, C₃-C₇ cycloalkyl, C₄-C₁₃ cycloalkylalkyl, heterocyclyl, aryl, and heteroaryl, wherein C₁-C₆ alkyl, C₃-C₇ cycloalkyl, C₄-C₁₃ cycloalkylalkyl, heterocyclyl, aryl, and heteroaryl can be optionally substituted from 1 to 3 times with a substituent selected independently at each occurrence from the group consisting of —OC₁-C₆ alkyl, —C(O)OH, and —C(O)OC₁-C₆ alkyl;
X is halogen, mesylate, tosylate, or triflate;
n is 0, 1, 2, 3, 4, 5, or 6;
m is 0, 1, 2, 3, 4, 5, 6, 7, 8, or 9; and
p is 0, 1, 2, 3, or 4;
or a solvate thereof,
with the proviso that when n is 1; R¹ is Me; m is 0; and p is 0, R² is not Ph.

Another aspect of the present disclosure relates to a compound of Formula (II):

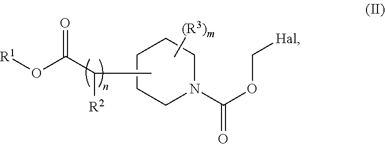

wherein
Hal is halogen;
R¹ is H or C₁-C₆ alkyl;
R² is independently selected from the group consisting of H, C₁-C₆ alkyl, C₃-C₇ cycloalkyl, C₄-C₁₃ cycloalkylalkyl, heterocyclyl, aryl, and heteroaryl, wherein C₁-C₆ alkyl, C₃-C₇ cycloalkyl, C₄-C₁₃ cycloalkylalkyl, heterocyclyl, aryl, and heteroaryl can be optionally substituted from 1 to 3 times with a substituent selected independently at each occurrence from the group consisting of C₁-C₆ alkyl, OH, halogen, and C₁-C₆ alkoxy;
R³ is independently selected at each occurrence thereof from the group consisting of H, C₁-C₆ alkyl, C₃-C₇ cycloalkyl, C₄-C₁₃ cycloalkylalkyl, heterocyclyl, aryl, and heteroaryl;
n is 0, 1, 2, 3, 4, 5, or 6; and
m is 0, 1, 2, 3, 4, 5, 6, 7, 8, or 9;
or a pharmaceutically acceptable salt thereof or a solvate thereof, with the proviso that when n is 1; R¹ is Me; m is 0; and p is 0, R² is not Ph.

The present disclosure involves preparation of aminomethyl carbamate compounds in the presence of a free carboxyl group by using excess alkali. The carboxyl group forms salt with alkali and does not participate in reaction with chloromethyl chloroformate.

### DETAILED DESCRIPTION

One aspect of the present disclosure relates to a process for preparation of a compound of Formula (I):

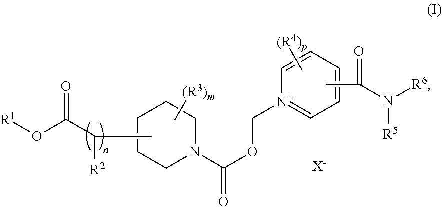

wherein
R¹ is C₁-C₆ alkyl;
R² is independently selected from the group consisting of H, C₁-C₆ alkyl, C₃-C₇ cycloalkyl, C₄-C₁₃ cycloalkylalkyl, heterocyclyl, aryl, and heteroaryl, wherein C₁-C₆ alkyl, C₃-C₇ cycloalkyl, C₄-C₁₃ cycloalkylalkyl, heterocyclyl, aryl, and heteroaryl can be optionally substituted from 1 to 3 times with a substituent selected independently at each occurrence from the group consisting of C₁-C₆ alkyl, OH, halogen, and C₁-C₆ alkoxy;
R³ is independently selected at each occurrence thereof from the group consisting of H, C₁-C₆ alkyl, C₃-C₇ cycloalkyl, C₄-C₁₃ cycloalkylalkyl, heterocyclyl, aryl, and heteroaryl;
R⁴ is independently selected at each occurrence thereof from the group consisting of H, C₁-C₆ alkyl, C₃-C₇ cycloalkyl, C₄-C₁₃ cycloalkylalkyl, heterocyclyl, aryl, and heteroaryl;
R⁵ is selected from the group consisting of H, C₁-C₆ alkyl, C₃-C₇ cycloalkyl, C₄- C₁₃ cycloalkylalkyl, heterocyclyl, aryl, and heteroaryl, wherein C₁-C₆ alkyl, C₃-C₇ cycloalkyl, C₄-C₁₃ cycloalkylalkyl, heterocyclyl, aryl, and heteroaryl can be optionally substituted from 1 to 3 times with a substituent selected independently at each occurrence from the group consisting of —OC₁-C₆ alkyl, —C(O)OH, and —C(O)OC₁-C₆ alkyl;
R⁶ is selected from the group consisting of H, C₁-C₆ alkyl, C₃-C₇ cycloalkyl, C₄- C₁₃ cycloalkylalkyl, heterocyclyl, aryl, and heteroaryl, wherein C₁-C₆ alkyl, C₃-C₇ cycloalkyl, C₄-C₁₃ cycloalkylalkyl, heterocyclyl, aryl, and heteroaryl can be optionally substituted from 1 to 3 times with a substituent selected independently at each occurrence from the group consisting of —OC₁-C₆ alkyl, —C(O)OH, and —C(O)OC₁-C₆ alkyl;
X is halogen, mesylate, tosylate, or triflate;
n is 0, 1, 2, 3, 4, 5, or 6;
m is 0, 1, 2, 3, 4, 5, 6, 7, 8, or 9; and
p is 0, 1, 2, 3, or 4;
   or a solvate thereof. This process includes:
providing an intermediate compound of Formula (II) having the structure:

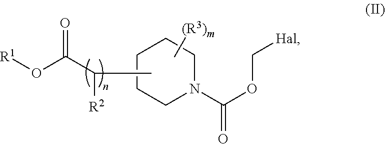

wherein Hal is halogen; and
forming the compound of Formula (I) from the intermediate compound of Formula (II).

As used above, and throughout the description herein, the following terms, unless otherwise indicated, shall be understood to have the following meanings. If not defined otherwise herein, all technical and scientific terms used herein have the same meaning as is commonly understood by one of ordinary skill in the art to which this technology belongs. In the event that there is a plurality of definitions for a term herein, those in this section prevail unless stated otherwise.

In this specification and the appended claims, the singular forms “a,” “an,” and “the” include plural references unless the context clearly dictates otherwise.

The terms “comprising,” “comprises,” and “comprised of” as used herein are synonymous with “including,” “includes,” or “containing,” “contains,” and are inclusive or open- ended and do not exclude additional, non-recited members, elements, or method steps.

The terms “comprising,” “comprises,” and “comprised of” also encompass the term “consisting of.” The transitional term “comprising,” which is synonymous with “including,” “containing,” or “characterized by,” is inclusive or open-ended and does not exclude additional, un-recited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed subject matter. In some embodiments or claims where the term comprising is used as the transition phrase, such embodiments can also be envisioned with replacement of the term “comprising” with the terms “consisting of” or “consisting essentially of.”

Terms of degree such as “substantially,” “about,” and “approximately” and the symbol “˜” as used herein mean a reasonable amount of deviation of the modified term such that the end result is not significantly changed. These terms of degree should be construed as including a deviation of at least ±0.1% (and up to ±1%, ±5%, or ±10%) of the modified term if this deviation would not negate the meaning of the word it modifies. Unless otherwise clear from context, all numerical values provided herein are modified by the term about. All numerical values provided herein that are modified by terms of degree set forth in this paragraph (e.g., “substantially,” “about,” “approximately,” and “˜”) are also explicitly disclosed without the term of degree. For example, “about 1%” is also explicitly disclosed as “1%”.

The term “and/or” as used herein means that the listed items are present, or used, individually or in combination. In effect, this term means that “at least one of” or “one or more” of the listed items is used or present.

The recitation of numerical ranges by endpoints includes all numbers and fractions subsumed within the respective ranges, as well as the recited endpoints. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member.

The term “alkyl” means an aliphatic hydrocarbon group which may be straight or branched having about 1 to about 10 carbon atoms in the chain. For example, straight or branched carbon chain could have 1 to 6 carbon atoms. Branched means that one or more lower alkyl groups such as methyl, ethyl, or propyl are attached to a linear alkyl chain. Exemplary alkyl groups include methyl, ethyl, n-propyl, i-propyl, n-butyl, t-butyl, n-pentyl, and 3-pentyl.

The term “cycloalkyl” means a non-aromatic mono-or multicyclic ring system of about 3 to about 7 carbon atoms, about 3 to about 6 carbon atoms, preferably of about 5 to about 7 carbon atoms. Exemplary monocyclic cycloalkyls include cyclopropyl, cyclobutyl, cyclopentyl, cyclohexyl, cycloheptyl, and the like.

The term “cycloalkylalkyl” means a cycloalkyl-alkyl-group in which the cycloalkyl and alkyl are as defined herein. Exemplary cycloalkylalkyl groups include cyclopropylmethyl, cyclobutylmethyl, cyclopentylmethyl, cyclopropylethyl, cyclobutylethyl, and cyclopentylethyl. The alkyl radical and the cycloalkyl radical may be optionally substituted as defined herein.

The term “aryl” means an aromatic monocyclic or multi-cyclic ring system of 6 to about 14 carbon atoms, preferably of 6 to about 10 carbon atoms. Representative aryl groups include, without limitation, phenyl and naphthyl.

The term “heteroaryl” means an aromatic monocyclic or multi-cyclic ring system of about 5 to about 14 ring atoms, in which one or more of the atoms in the ring system is/are clement(s) other than carbon, for example, nitrogen, oxygen, or sulfur. In some embodiments, a “heteroaryl” is an aromatic monocyclic or multi-cyclic ring system of about 5 to about 10 ring atoms. In the case of multi-cyclic ring system, only one of the rings needs to be aromatic for the ring system to be defined as “heteroaryl.” In some embodiments, exemplary heteroaryls contain about 5 to 6 ring atoms. The prefix aza, oxa, thia, or thio before heteroaryl means that at least a nitrogen, oxygen, or sulfur atom, respectively, is present as a ring atom. A nitrogen atom of a heteroaryl is optionally oxidized to the corresponding N-oxide. Representative heteroaryls include, without limitation, pyridyl, 2-oxo-pyridinyl, pyrimidinyl, pyridazinyl, pyrazinyl, triazinyl, furanyl, pyrrolyl, thiophenyl, pyrazolyl, imidazolyl, oxazolyl, isoxazolyl, thiazolyl, isothiazolyl, triazolyl, oxadiazolyl, thiadiazolyl, tetrazolyl, indolyl, isoindolyl, benzofuranyl, benzothiophenyl, indolinyl, 2-oxoindolinyl, dihydrobenzofuranyl, dihydrobenzothiophenyl, indazolyl, benzimidazolyl, benzooxazolyl, benzothiazolyl, benzoisoxazolyl, benzoisothiazolyl, benzotriazolyl, benzo[1,3]dioxolyl, quinolinyl, isoquinolinyl, quinazolinyl, cinnolinyl, pthalazinyl, quinoxalinyl, 2,3-dihydro-benzo[1,4]dioxinyl, benzo[1,2,3]triazinyl, benzo[1,2,4]triazinyl, 4H-chromenyl, indolizinyl, quinolizinyl, 6aH-thieno[2,3-d]imidazolyl, 1H-pyrrolo[2,3-b]pyridinyl, imidazo[1,2-a]pyridinyl, pyrazolo[1,5-a]pyridinyl, [1,2,4]triazolo[4,3-alpyridinyl, [1,2,4]triazolo[1,5-a]pyridinyl, thieno[2,3-b]furanyl, thieno[2,3-b]pyridinyl, thieno[3,2-b]pyridinyl, furo 8 2,3-b]pyridinyl, furo[3,2-b]pyridinyl, thieno[3,2-d]pyrimidinyl, furo[3,2-d]pyrimidinyl, thieno[2,3-b]pyrazinyl, imidazo[1,2-a]pyrazinyl, 5,6,7,8-tetrahydroimidazo[1,2-a]pyrazinyl, 6,7-dihydro-4H-pyrazolo[5,1-c][1,4]oxazinyl, 2-oxo-2,3-dihydrobenzo[d]oxazolyl, 3,3-dimethyl-2-oxoindolinyl, 2-oxo-2,3-dihydro-1 H-pyrrolo [2,3-b]pyridinyl, benzo[c][1,2,5]oxadiazolyl, benzo[c][1,2,5]thiadiazolyl, 3,4-dihydro-2H-benzo[b][1,4]oxazinyl, 5,6,7,8-tetrahydro-[1,2,4]triazolo[4,3-a]pyrazinyl, [1,2,4]triazolo[4,3-a]pyrazinyl, 3-oxo-[1,2,4]triazolo[4,3-a]pyridin-2(3H)-yl, and the like.

As used herein, “heterocyclyl” or “heterocycle” refers to a stable 3- to 18-membered ring (radical) which consists of carbon atoms and from one to five heteroatoms selected from the group consisting of nitrogen, oxygen, and sulfur. For purposes of this application, the heterocycle may be a monocyclic, or a polycyclic ring system, which may include fused, bridged, or spiro ring systems; and the nitrogen, carbon, or sulfur atoms in the heterocycle may be optionally oxidized; the nitrogen atom may be optionally quaternized; and the ring may be partially or fully saturated. Examples of such heterocycles include, without limitation, oxiranyl, azepinyl, azocanyl, pyranyl dioxanyl, dithianyl, 1,3-dioxolanyl, tetrahydrofuryl, dihydropyrrolidinyl, decahydroisoquinolyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, 2-oxoazepinyl, oxazolidinyl, oxiranyl, piperidinyl, piperazinyl, 4-piperidonyl, pyrrolidinyl, pyrazolidinyl, thiazolidinyl, tetrahydropyranyl, thiamorpholinyl, thiamorpholinyl sulfoxide, and thiamorpholinyl sulfone. Further heterocycles and heteroaryls are described in Katritzky et al., eds., Comprehensive Heterocyclic Chemistry: The Structure, Reactions, Synthesis and Use of Heterocyclic Compounds, Vol. 1-8, Pergamon Press, N. Y. (1984), which is hereby incorporated by reference in its entirety.

The term “monocyclic” used herein indicates a molecular structure having one ring.

The term “polycyclic” or “multi-cyclic” used herein indicates a molecular structure having two or more rings, including, but not limited to, fused, bridged, or spiro rings.

The term “halo” or “halogen” means fluoro, chloro, bromo, or iodo.

The term “alkoxy” means groups of from 1 to 12 carbon atoms of a straight, branched, or cyclic configuration and combinations thereof attached to the parent structure through an oxygen. Examples include methoxy, ethoxy, propoxy, isopropoxy, cyclopropyloxy, cyclohexyloxy, and the like. Lower-alkoxy refers to groups containing one to four carbons. For the purposes of the present patent application, alkoxy also includes methylenedioxy and ethylenedioxy in which each oxygen atom is bonded to the atom, chain, or ring from which the methylenedioxy or ethylenedioxy group is pendant so as to form a ring. Thus, for example, phenyl substituted by alkoxy may be, for example,

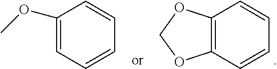

The term “substituted” or “substitution” of an atom means that one or more hydrogen on the designated atom is replaced with a selection from the indicated group, provided that the designated atom's normal valency is not exceeded.

The term “optionally substituted” is used to indicate that a group may have a substituent at each substitutable atom of the group (including more than one substituent on a single atom), provided that the designated atom's normal valency is not exceeded, and the identity of each substituent is independent of the others. Up to three H atoms in each residue are replaced with alkyl, halogen, haloalkyl, hydroxy, loweralkoxy, carboxy, carboalkoxy (also referred to as alkoxycarbonyl), carboxamido (also referred to as alkylaminocarbonyl), cyano, carbonyl, nitro, amino, alkylamino, dialkylamino, mercapto, alkylthio, sulfoxide, sulfone, acylamino, amidino, phenyl, benzyl, heteroaryl, phenoxy, benzyloxy, or heteroaryloxy.

“Unsubstituted” atoms bear all of the hydrogen atoms dictated by their valency. When a substituent is keto (i.e., ═O), then two hydrogens on the atom are replaced. Combinations of substituents and/or variables are permissible only if such combinations result in stable compounds; by “stable compound” or “stable structure” is meant a compound that is sufficiently robust to survive isolation to a useful degree of purity from a reaction mixture, and formulation into an efficacious therapeutic agent.

Compounds described herein may contain one or more asymmetric centers and may thus give rise to enantiomers, diastereomers, and other stereoisomeric forms. Each chiral center may be defined, in terms of absolute stereochemistry, as (R)- or(S)-. This technology is meant to include all such possible isomers, as well as mixtures thereof, including racemic and optically pure forms. Optically active (R)- and(S)-, (−)- and (+)-, or (D)- and (L)-isomers may be prepared using chiral synthons or chiral reagents, or resolved using conventional techniques.

In some embodiments, C₃-C₇ cycloalkyl is C₃-C₆ cycloalkyl.

In some embodiments, n is 0. In other embodiments, n is 1. In other embodiments, n is 2. In yet other embodiments, n is 3. In some other embodiments, n is 4. In other embodiments, n is 5. In other embodiments, n is 6.

In some embodiments, R¹ is methyl.

In some embodiments, R² is independently selected from the group consisting of

C₁-C₆ alkyl, C₃-C₇ cycloalkyl, C₄-C₁₃ cycloalkylalkyl, heterocyclyl, aryl, and heteroaryl, wherein C₁-C₆ alkyl, C₃-C₇ cycloalkyl, C₄-C₁₃ cycloalkylalkyl, heterocyclyl, aryl, and heteroaryl can be optionally substituted from 1 to 3 times with a substituent selected independently at each occurrence from the group consisting of C₁-C₆ alkyl, OH, halogen, and C₁-C₆ alkoxy.

In some embodiments, R⁵ is H.

In some embodiments, the compound of Formula (I) has the structure selected from the group consisting of

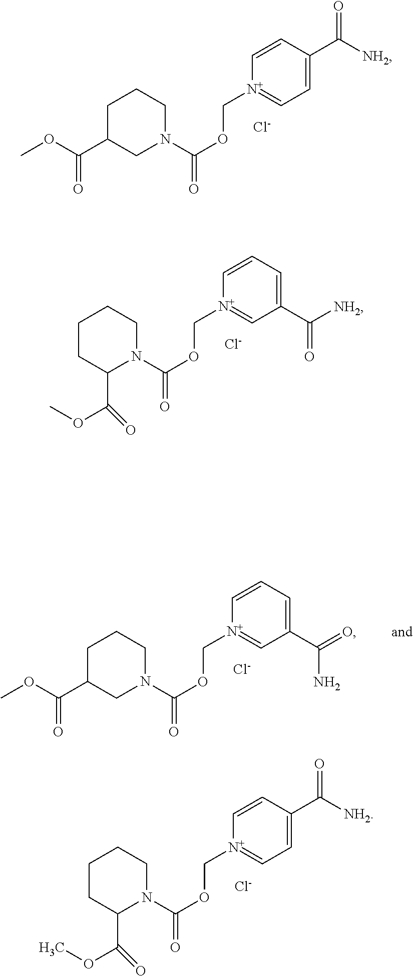

In some embodiments, the compound of Formula (I) has the following structure:

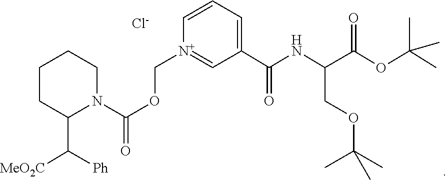 [SMILES: COC(=O)C(c1ccccc1)C1CCCCN1C(=O)OC[n+]1cccc(C(=O)NC(COC(C)(C)C)C(=O)OC(C)(C)C)c1 ; [Cl-]]

In some embodiments, the compound of Formula (I) has the following structure:

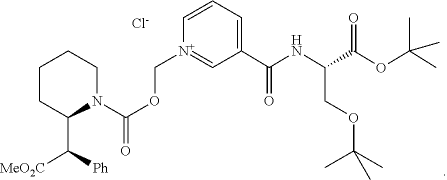 [SMILES: COC(=O)[C@H](c1ccccc1)[C@H]1CCCCN1C(=O)OC[n+]1cccc(C(=O)N[C@@H](COC(C)(C)C)C(=O)OC(C)(C)C)c1 ; [Cl-]]

In some embodiments, the compound of Formula (II) has the structure selected from the group consisting of:

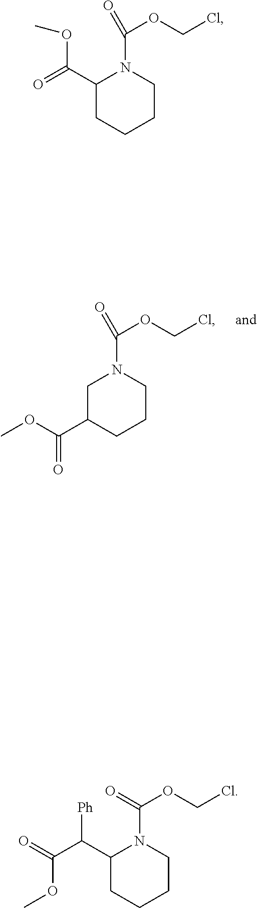 [SMILES: COC(=O)C1CCCCN1C(=O)OCCl ; COC(=O)C1CCCN(C(=O)OCCl)C1 ; COC(=O)C(c1ccccc1)C1CCCCN1C(=O)OCCl]

In some embodiments, the compound of Formula (II) is

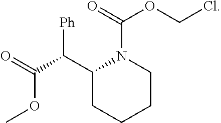 [SMILES: COC(=O)[C@H](c1ccccc1)[C@H]1CCCCN1C(=O)OCCl]

In some embodiments, forming the compound of Formula (I) comprises:
reacting the intermediate compound of Formula (II) with a compound of Formula (III):

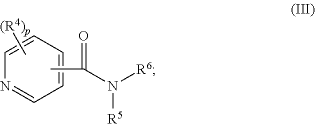

to produce the compound of Formula (I).

In some embodiments, the compound of Formula (III) has the structure selected from the group consisting of:

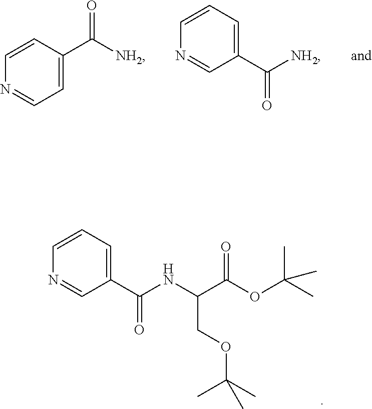 [SMILES: NC(=O)c1ccncc1 ; NC(=O)c1cccnc1 ; "and" ; CC(C)(C)OCC(NC(=O)c1cccnc1)C(=O)OC(C)(C)C]

In some embodiments, the compound of Formula (III) is

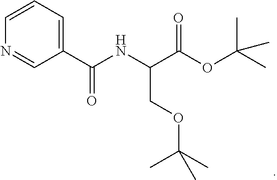 [SMILES: CC(C)(C)OCC(NC(=O)c1cccnc1)C(=O)OC(C)(C)C]

In some embodiments, the step of reacting the intermediate compound of Formula (II) with the compound of Formula (III) is carried out at a temperature of from about 25° C. to about 80° C., from about 30° C. to about 75° C., from about 35° C. to about 75° C., from about 40° C. to about 75° C., from about 45° C. to about 75° C., from about 45° C. to about 70° C., from about 50° C. to about 70° C., from about 55° C. to about 70° C., or from about 55° C. to about 65° C.

In some embodiments, the process further includes:
providing an intermediate compound of Formula (IVa) and/or Formula (IVb):

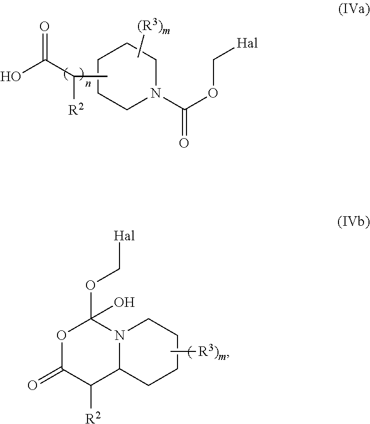

and
forming the intermediate compound of Formula (II) from the intermediate compound of Formula (IVa) and/or Formula (IVb).

In some embodiments, the forming of the intermediate compound of Formula (II) comprises:
reacting the intermediate compound of Formula (IVa) with an alkylating agent to produce the intermediate compound of Formula (II).

In some embodiments, the forming of the intermediate compound of Formula (II) comprises:
reacting the intermediate compound of Formula (IVb) with an alkylating agent to produce the intermediate compound of Formula (II).

In some embodiments, the forming of the intermediate compound of Formula (II) comprises:
reacting a mixture of the intermediate compound of Formula (IVa) and Formula (IVb) with an alkylating agent to produce the intermediate compound of Formula (II).

According to the present disclosure, the alkylating agent in any of the above embodiments can be SOCl₂/C₁₋₆ alkyl-OH (e.g., SOCl₂/MeOH, SOCl₂/EtOH, SOCl₂/PrOH, SOCl₂/i-PrOH, SOCl₂/BuOH, SOCl₂/i-BuOH, etc.). In some embodiments, the alkylating agent is SOCl₂/MeOH.

In some embodiments, the intermediate compound of Formula (IVa) has a structure selected from the group consisting of:

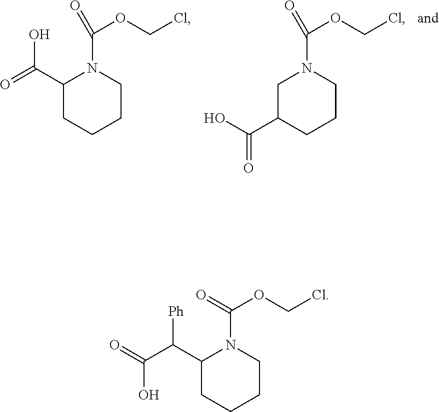 [SMILES: O=C(O)C1CCCCN1C(=O)OCCl ; O=C(O)C1CCCN(C(=O)OCCl)C1 ; O=C(O)C(c1ccccc1)C1CCCCN1C(=O)OCCl]

In some embodiments, the intermediate compound of Formula (IVa) is

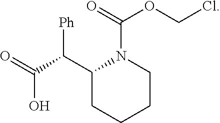 [SMILES: O=C(O)[C@H](c1ccccc1)[C@H]1CCCCN1C(=O)OCCl]

In some embodiments, the intermediate compound of Formula (IVb) is

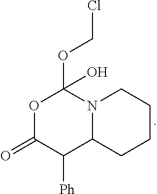 [SMILES: O=C1OC(O)(OCCl)N2CCCCC2C1c1ccccc1]

In some embodiments, the intermediate compound of Formula (IVb) is

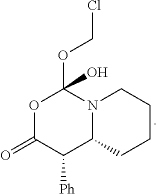 [SMILES: O=C1O[C@](O)(OCCl)N2CCCC[C@@H]2[C@@H]1c1ccccc1]

In some embodiments, the method further comprises:
providing a compound of Formula (V):

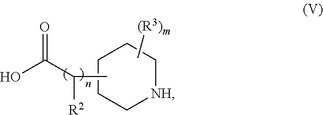

and
forming the intermediate compound of Formula (IVa) and/or (IVb) from the compound of Formula (V).

In some embodiments, forming the intermediate compound of Formula (IVa) and/or (IVb) comprises:
reacting the compound of Formula (V) with a compound of Formula (VI):

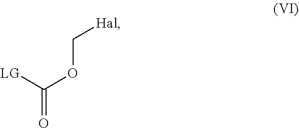

wherein LG is a suitable leaving group. Suitable living group (LG) that can be used include, but not limited to halogen, benzotriazolyl (Bt), or acyloxy.

In some embodiments, the compound of Formula (VI) is chloromethyl chloroformate.

In some embodiments, the step of reacting the compound of Formula (V) with the compound of Formula (VI) is carried out in the presence of a base. Suitable bases that can be used include, but are not limited to NaOH, KOH, LiOH, CsOH, Ca(OH)₂, and Mg(OH)_{2.}

In some embodiments, the step of reacting the compound of Formula (V) with the compound of Formula (VI) can carried out at a temperature of from about 5° C. to about 25° C., from about 5° C. to about 20° C., from about 10° C. to about 20° C., from about 15° C. to about 20° C., from about 11° C. to about 19° C., from about 12° C. to about 18° C., from about 13° C. to about 17° C., or from about 14° C. to about 16° C.

In some embodiments, the step of reacting the compound of Formula (V) with the compound of Formula (VI) is carried out at a temperature of about 10° C., 11° C., 12° C., 13° C., 14° C., 15° C., 16° C., 17° C., 18° C., 19° C., or 20° C.

In some embodiments, the compound of Formula (V) is selected from the group consisting of:

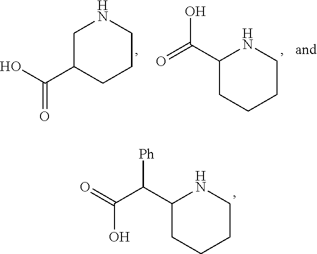

or a salt thereof.

In some embodiments, the compound of Formula (V) is

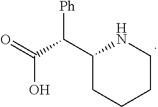 [SMILES: O=C(O)[C@H](c1ccccc1)[C@H]1CCCCN1]

Another aspect of the present disclosure relates to a compound of Formula (I):

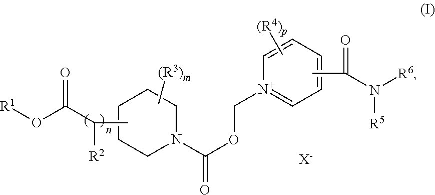

wherein
R¹ is C₁-C₆ alkyl;
R² is independently selected from the group consisting of H, C₁-C₆ alkyl, C₃-C₇ cycloalkyl, C₄-C₁₃ cycloalkylalkyl, heterocyclyl, aryl, and heteroaryl, wherein C₁-C₆ alkyl, C₃-C₇ cycloalkyl, C₄-C₁₃ cycloalkylalkyl, heterocyclyl, aryl, and heteroaryl can be optionally substituted from 1 to 3 times with a substituent selected independently at each occurrence from the group consisting of C₁-C₆ alkyl, OH, halogen, and C₁-C₆ alkoxy;
R³ is independently selected at each occurrence thereof from the group consisting of H, C₁-C₆ alkyl, C₃-C₇ cycloalkyl, C₄-C₁₃ cycloalkylalkyl, heterocyclyl, aryl, and heteroaryl;
R⁴ is independently selected at each occurrence thereof from the group consisting of H, C₁-C₆ alkyl, C₃-C₇ cycloalkyl, C₄-C₁₃ cycloalkylalkyl, heterocyclyl, aryl, and heteroaryl;
R⁵ is selected from the group consisting of H, C₁-C₆ alkyl, C₃-C₇ cycloalkyl, C₄-C₁₃ cycloalkylalkyl, heterocyclyl, aryl, and heteroaryl, wherein C₁-C₆ alkyl, C₃-C₇ cycloalkyl, C₄-C₁₃ cycloalkylalkyl, heterocyclyl, aryl, and heteroaryl can be optionally substituted from 1 to 3 times with a substituent selected independently at each occurrence from the group consisting of —OC₁-C₆ alkyl,-C (O) OH, and-C (O) OC₁-C₆ alkyl;
R⁶ is selected from the group consisting of H, C₁-C₆ alkyl, C₃-C₇ cycloalkyl, C₄-C₁₃ cycloalkylalkyl, heterocyclyl, aryl, and heteroaryl, wherein C₁-C₆ alkyl, C₃-C₇ cycloalkyl, C₄-C₁₃ cycloalkylalkyl, heterocyclyl, aryl, and heteroaryl can be optionally substituted from 1 to 3 times with a substituent selected independently at each occurrence from the group consisting of —OC₁-C₆ alkyl,-C (O) OH, and-C (O) OC₁-C₆ alkyl;
X is halogen, mesylate, tosylate, or triflate;
n is 0, 1, 2, 3, 4, 5, or 6;
m is 0, 1, 2, 3, 4, 5, 6, 7, 8, or 9;
p is 0, 1, 2, 3, or 4;
or a solvate thereof,
   with the proviso that when n is 1; R¹ is Me; m is 0; and p is 0, R² is not Ph.

In some embodiments, R² is independently selected from the group consisting of C₁-C₆ alkyl, C₃-C₇ cycloalkyl, C₄-C₁₃ cycloalkylalkyl, heterocyclyl, aryl, and heteroaryl, wherein C₁-C₆ alkyl, C₃-C₇ cycloalkyl, C₄-C₁₃ cycloalkylalkyl, heterocyclyl, aryl, and heteroaryl can be optionally substituted from 1 to 3 times with a substituent selected independently at each occurrence from the group consisting of C₁-C₆ alkyl, OH, halogen, and C₁-C₆ alkoxy.

Another aspect of the present disclosure relates to a compound of Formula (II):

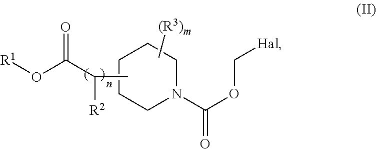

and wherein
Hal is halogen;
R¹ is H or C₁-C₆ alkyl;
R² is independently selected from the group consisting of H, C₁-C₆ alkyl, C₃-C₇ cycloalkyl, C₄-C₁₃ cycloalkylalkyl, heterocyclyl, aryl, and heteroaryl, wherein C₁-C₆ alkyl, C₃-C₇ cycloalkyl, C₄-C₁₃ cycloalkylalkyl, heterocyclyl, aryl, and heteroaryl can be optionally substituted from 1 to 3 times with a substituent selected independently at each occurrence from the group consisting of C₁-C₆ alkyl, OH, halogen, and C₁-C₆ alkoxy;
R³ is independently selected at each occurrence thereof from the group consisting of H, C₁-C₆ alkyl, C₃-C₇ cycloalkyl, C₄-C₁₃ cycloalkylalkyl, heterocyclyl, aryl, and heteroaryl;
n is 0, 1, 2, 3, 4, 5, or 6; and
m is 0, 1, 2, 3, 4, 5, 6, 7, 8, or 9;
or a pharmaceutically acceptable salt thereof or a solvate thereof,
   with the proviso that when n is 1; R¹ is Me; m is 0; and p is 0, R² is not Ph.

In some embodiments, R¹ is C₁-C₆ alkyl. In other embodiments, R¹ is H.

In some embodiments, R² is independently selected from the group consisting of C₁-C₆ alkyl, C₃-C₇ cycloalkyl, C₄-C₁₃ cycloalkylalkyl, heterocyclyl, aryl, and heteroaryl, wherein C₁-C₆ alkyl, C₃-C₇ cycloalkyl, C₄-C₁₃ cycloalkylalkyl, heterocyclyl, aryl, and heteroaryl can be optionally substituted from 1 to 3 times with a substituent selected independently at each occurrence from the group consisting of C₁-C₆ alkyl, OH, halogen, and C₁-C₆ alkoxy.

The above disclosure is general. A more specific description is provided below in the following examples. The examples are described solely for the purpose of illustration and are not intended to limit the scope of the present application. Changes in form and substitution of equivalents are contemplated as circumstances suggest or render expedient. Although specific terms have been employed herein, such terms are intended in a descriptive sense and not for purposes of limitation.

### EXAMPLES

The following Examples are presented to illustrate various aspects of the present disclosure, but are not intended to limit the scope of the claimed application.

### Example 1—Preparation of Aminomethyl Carbamate Using Piperidine 3-Carboxylicacid and Isonicotinamide

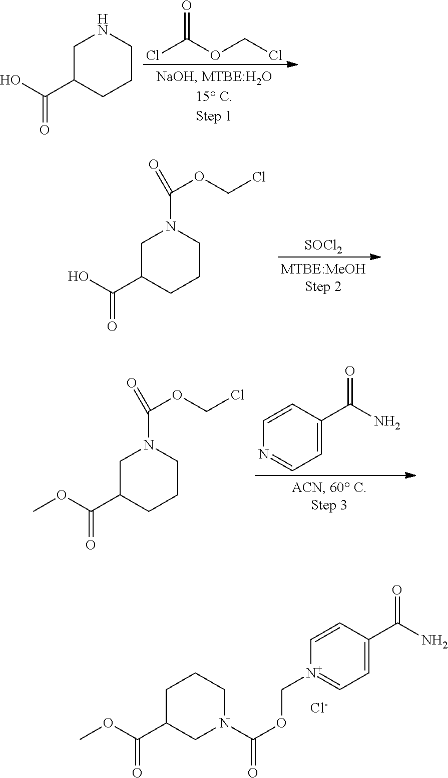

Step 1: Preparation of Chloromethyl Carbamate with Piperidine-3-Carboxylic Acid

Piperidine-3-carboxylic acid (5.0 g., 0.0387 mol., 1.0 eq.) was added to an oven dried 4 necked round-bottom flask (RBF) at room temperature (25° C.). Methyl tert-butyl ether (MTBE) (25 mL, 5 vol) was added at 25° C. to the above RBF. The solution was stirred at 20-25° C. and a solution of NaOH (3.0 gm, 0.0774 mol., 2 eq.) in 20 ml (4 vol) of water was added dropwise through dropping funnel. The above reaction mixture stirred at 20-25° C. for 20 min. and then cooled to 15° C. Chloromethyl chloroformate (CMCF) (9.98 g., 0.0774 mol., 2 eq.) was added dropwise. During addition of CMCF, pH was maintained between 8.5 to 9.5 by addition of a solution of NaOH (4.6 gm, 0.1161 mol., 3 eq.) in 10 mL of water (2 vol) at 15-20° C. After addition was completed, reaction mixture was stirred for 1 hour at 15-20° C. (pH 8.5-9.5 was maintained with addition of above NaOH solution during the charge of CMCF). Reaction progress was monitored by HPLC. After consumption of starting material, MTBE (5 mL, 1 vol) was added and the reaction mixture was stirred for 20 min at 20-25° C. The aqueous layer was separated. Water (10 mL, 2 vol) was added to the organic layer and the mixture was stirred for 20 min at 25° C. The aqueous layer was separated. The organic layer was concentrated under reduced pressure to obtain crude carbamate product (2.6 g). Crude product was used in next step without further purification.

Step 2: Preparation of Methyl Ester of Chloromethyl Carbamate of Piperidine-3-carboxylic Acid

Crude carbamate (2.5 g., 1.0 eq., 0.011 mol) was added to a 4-necked RBF at 25° C. under N₂. MTBE (15 mL, 6 vol) and methanol (5 mL, 2 vol) were added at 25° C. under N₂ to the above RBF. Thionyl chloride (1.60 mL, 2.0 eq., 0.022 mol.) was slowly added at 20-25° C. The reaction was then stirred at room temperature for 14-16 hours. Reaction progress was monitored by HPLC. After consumption of starting material, reaction mixture was concentrated under reduced pressure at 40° C. The flask was cooled to 5-10° C. and 10 mL (4 vol) of purified water and 10 mL (4 vol) of MTBE were added. The reaction mixture was stirred for 2 hours. The aqueous layer was separated. Saturated sodium bicarbonate solution (10 mL, 4 vol) was added to the organic layer and the mixture was stirred for 2 hours at 10-15° C. The aqueous layer was separated. The organic layer was concentrated under reduced pressure to get methyl ester of chloromethyl carbamate (2.1 g). Crude product was dried over rotavapor and used in next step without further purification.

### Step 3: Preparation of Aminomethyl Carbamate Using Piperidine 3-Carboxylicacid and Isonicotinamide

Acetonitrile (16 mL, 8 vol) was added to crude methyl ester of chloromethyl carbamate (2 g, 1.0 eq., 0.0084 mol.) at 25° C. under N₂ in a 4 necked dried RBF. Acetonitrile was subsequently distilled at 60° C. up to 4 vol. Reaction mixture was cooled to 35-40° C. Isonicotinamide (0.82 g, 0.8 eq., 0.006 mol.) was added portion wise to the above solution at 35-40° C. under N₂. The reaction mixture was heated at 60° C. and stirred for 22-24 hours. Reaction progress was monitored by HPLC. After consumption of starting material, reaction mixture was cooled to 25° C. n-Heptane (28 mL, 14 vol) and methyl isobutyl ketone (MIBK) (14 mL, 7 vol) were added to a separate 100 mL RBF, and stirred at 20° C. for 10 min. Reaction mixture was added to the above RBF slowly at 20° C., resulting in the formation of a slurry. The slurry was stirred at 20° C. for 5-6 hours. Suspension was filtered under N₂. The wet cake was washed by a cold solution of n-heptane (4 mL, 2 vol) and MIBK (2 mL, 1 vol) under N₂. Purification by silica gel combi-flash column chromatography followed by recrystallization in dichloromethane and isopropyl acetate gave the product (2.4 g) (17.4% of three steps overall yield, HPLC purity 96%).

### Characterization of Aminomethyl Carbamate Prepared Using Piperidine-3-Carboxylic Acid and Isonicotinamide

The product exists as rotational isomers (rotamers) due to the restricted rotation around the carbamate functional group. Due to this, more ¹H and C¹³ NMR signals are seen than can be accounted for the structure. To demonstrate this fact, ¹H NMR was performed at 90° C. where the rotamers collapsed and provided the 1H-NMR peaks that account for the structure (Table 1).

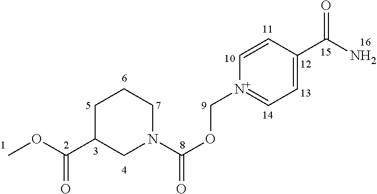 [SMILES: COC(=O)C1CCCN(C(=O)OC[n+]2ccc(C(N)=O)cc2)C1]

TABLE 1
Proton NMR at 90° C. in DMSO-d₆

| Proton # | δ (ppm) | Integration |
|---|---|---|
| 10, 14 | 9.38 | 1.90, 2H |
| 11, 13 | 8.58 | 2.01, 2H |
| 16 | 7.9-9.0, broad | 1.83, 2H |
| 9 | 6.5 | 1.89, 2H |
| 4 | 3.88, 3.65 | 0.99, 0.83, 2H |
| 1 | 3.60 | 3.02, 3H |
| 7 | 3.28, 3.12 | 0.94, 1.18, 2H |
| 3 | 2.56 | 0.97, 1H |
| 5 | 1.92, 1.64 | 1.00, 0.99, 2H |
| 6 | 1.64, 1.46 | 0.99, 1.01, 2H |

C¹³ NMR (solvent CDCl₃) was performed at room temperature and there were more carbon peaks than accounted for the structure due to the presence of rotamers (Table 2).

TABLE 2
C¹³ NMR

| Carbon # | δ (ppm) |
|---|---|
| 2 | 173.24 |
| 15 | 163.52 |
| 8 | 152.94, 152.83 |
| 12 | 150.10 |
| 10 | 146.07 |
| 14 | 145.96 |
| 11 | 126.92 |
| 13 | 126.92 |
| 9 | 80.92 |
| 1 | 52.08, 51.96 |
| 7 | 45.96, 45.79 |
| 4 | 44.79, 44.61 |
| 3 | 40.65, 40.53 |
| 5 | 26.78 |
| 6 | 24.07, 23.46 |

IR (film) vmax 3360, 3140, 3100, 3000, 2950, 1720, 1650, 1600, 1560, 1450, cm⁻¹. UPLCMS m/z calculated for C₁₅H₂₀N₃O₅⁺ [M+H]⁺ 323.34, found 323.21.

### Example 2—Preparation of Aminomethyl Carbamate Using Piperidine-2-Carboxylicacid and Nicotinamide

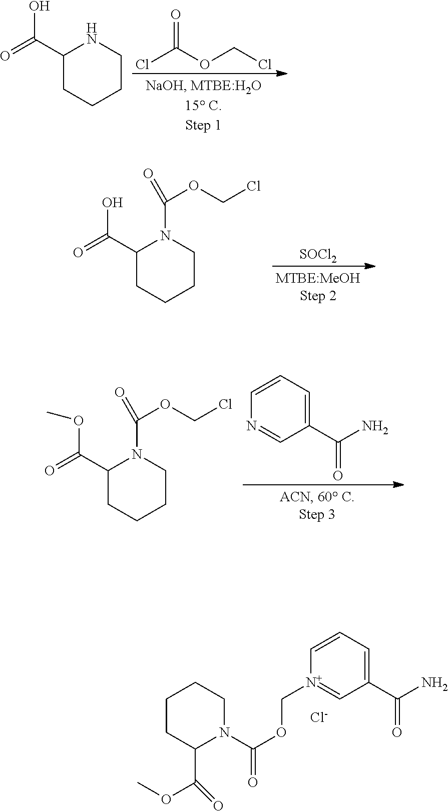

### Step 1: Preparation of Chloromethyl Carbamate With Piperidine-2-Carboxylic Acid

Charged 5.0 g of Piperidine-2-carboxylic acid (0.0387 mol., 1.0 eq.) in oven dried 4 necked RBF at room temperature (25° C.). Charged 25 mL of methyl tert-butyl ether (MTBE, 5 vol) at 25° C. to the above RBF. The solution was stirred at 20-25° C. and added a solution of NaOH (3.0 gm, 0.0774 mol., 2 eq.) in 20 ml (4 vol) of water dropwise through dropping funnel. The above reaction mixture stirred at 20-25° C. until clear solution. (˜1 hr.) Afterwards the reaction mixture cooled to 15° C. Chloromethyl chloroformate (CMCF, 9.98 gm, 0.0774 mol., 2.0 eq.) was added dropwise. During addition of CMCF, maintain pH between 8.5 to 9.5 by addition of solution of NaOH (4.6 gm, 0.1161 mol., 3 eq.) in 10 mL water (2 vol) at 15-20° C. After addition completed, reaction mixture was stirred for 1 hr. at 15-20° C. (Note: maintain pH 8.5-9.5 with addition of above NaOH solution). Reaction progress was monitored by HPLC. After consumption of starting material, added 5 mL (1 vol) of MTBE and stirred reaction mixture for 20 min at 20-25° C. Separated the aqueous layer. To the organic layer, added 10 mL water (2 vol) and stirred for 20 min at 25° C. Separated the aqueous layer. Organic layer concentrated under reduced pressure to obtain crude carbamate product, 2.7 gm. Crude product was used in next step without further purification.

### Step 2: Preparation of Methyl Ester of Chloromethyl Carbamate of Piperidine-2-Carboxylic Acid

Charged crude carbamate 2.5 g (1.0 eq., 0.011 mol.) at 25° C. under N₂ to a 4-necked RBF. Charged MTBE 16 mL (6 vol) and methanol 5.5 mL (2 vol) at 25° C. under N₂ to the above RBF. Thionyl chloride, 1.76 mL (2.0 eq., 0.022 mol.) was slowly added at 20-25° C. The reaction was then stirred at RT for 14-16 hours. Reaction progress was monitored by HPLC. After consumption of starting material, reaction mixture was concentrated under reduced pressure at 40° C. The flask was cooled to 5-10° C. and 10 mL (4 vol) of purified water and 10 mL (4 vol) of MTBE were added and stirred the reaction mixture for 2 hours. Separated the aqueous layer. To the organic layer, was added saturated sodium bicarbonate solution (10 mL, 4 vol) and stirred for 2 hours at 10-15°° C. Separated the aqueous layer. Organic layer was concentrated under reduced pressure to get crude methyl ester of carbamate, 2.5 gm. Crude product used in next step without further purification.

Step-3: Preparation of Aminomethyl Carbamate Using Piperidine-2-carboxylic Acid and Nicotinamide

Charged acetonitrile (20 mL, 8.0 vol) to Crude Methyl Ester of Carbamate (2.5 g, 1.0 eq., 0.0106 mol.) at 25° C. under N₂ in a 4 necked dried RBF. Acetonitrile was subsequently distilled at 60° C. up to 4 vol. Reaction mixture cooled to 35-40° C. Nicotinamide (1.03 g, 0.8 eq., 0.0084 mol.) was added portion wise to above solution at 40-45° C. under N₂. The reaction mixture was heated at 60° C. and stirred for 22-24 hours. Reaction progress was monitored by HPLC. After consumption of starting material, reaction was cooled to 25° C. n-Heptane, 35 mL (14 vol) and methyl isobutyl ketone, 17.5 mL (MIBK, 7 vol) were charged to a separate 100 mL RBF, and stirred at 20° C. for 10 min. Reaction mixture was added to the above RBF slowly at 20° C., resulting in formation of a slurry. The slurry was stirred at 20° C. for 5-6 hours. Suspension was filtered under N₂. The wet cake was washed by a cold solution of 5 mL of n-heptane (2 vol) and 2.5 mL of MIBK (1 vol) under N2 and dried by suction under vacuum. Purification by silica combi-flash column chromatography followed by recrystallization in dichloromethane and isopropyl acetate gave product (5.3 g) (38.4% overall yield for 3 steps). HPLC purity 98.40%.

Characterization of Aminomethyl Carbamate Prepared Using Piperidine-2-carboxylic Acid and Nicotinamide

The product exists as rotational isomers (rotamers) due to the restricted rotation around the carbamate functional group. Due to this more ¹H and C¹³ NMR signals are seen than can be accounted for the structure. To demonstrate this fact, ¹H NMR was performed at 90° C. where the rotamers collapsed and provided the ¹H-NMR peaks that account for the structure (Table 3).

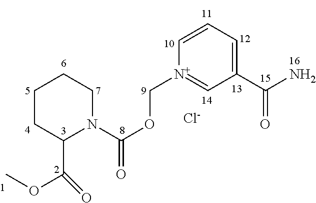 [SMILES: COC(=O)C1CCCCN1C(=O)OC[n+]1cccc(C(N)=O)c1 ; [Cl-]]

TABLE 3
Proton NMR at 90° C. in DMSO-d₆

| Proton # | δ (ppm) | Integration |
|---|---|---|
| 14 | 9.70 | 1.00, 1H |
| 12 | 9.28 | 1.00, 1H |
| 10 | 9.15 | 1.01, 1H |
| 16-NH₂ | 8.90-7.50, broad | 1.74, 2H |
| 11 | 8.30 | 1.16, 1H |
| 9 | 6.50 | 2.03, 2H |
| 3 | 4.86 | 1.01, 1H |
| 1 | 3.64 | 3.11, 3H |
| 7 | 3.90, 2.90 | 1.04, 0.92, 2H |
| 4 | 2.10, 1.70 | 1.03, 1.04, 2H |
| 6 | 1.62 | 2.10, 2H |
| 5 | 1.40, 1.20 | 1.21, 1.05, 2H |

C¹³ NMR (solvent DMSO-d6) was performed at room temperature and there were more peaks observed than accounted for the structure due to the presence of rotamers (Table 4).

TABLE 4
C¹³ NMR

| Carbon # | δ (ppm) |
|---|---|
| 2 | 171.28, 171.43 |
| 15 | 162.87 |
| 8 | 154.21, 153.82 |
| 12 | 147.00 |
| 14 | 146.54 |
| 13 | 145.64, 145.52 |
| 10 | 134.00, 133.91 |
| 11 | 128.33, 128.13 |
| 9 | 81.14, 80.73 |
| 3 | 55.12, 54.79 |
| 1 | 53.02, 52.89 |
| 7 | 42.51, 42.39 |
| 4 | 26.51, 26.31, |
| 6 | 24.35, 24.16 |
| 5 | 20.43 |

IR (film) vmax 3220, 3120, 3050, 2950, 2900, 1750, 1725,1700,1620,1450, 1400 cm⁻¹. UPLCMS m/z calculated for C₁₅H₂₀N₃O₅⁺ [M+H]⁺ 323.34, found 323.25.

### Example 3—Preparation of Aminomethyl Carbamate Synthon Using Piperidine-3-Carboxylic Acid and Nicotinamide

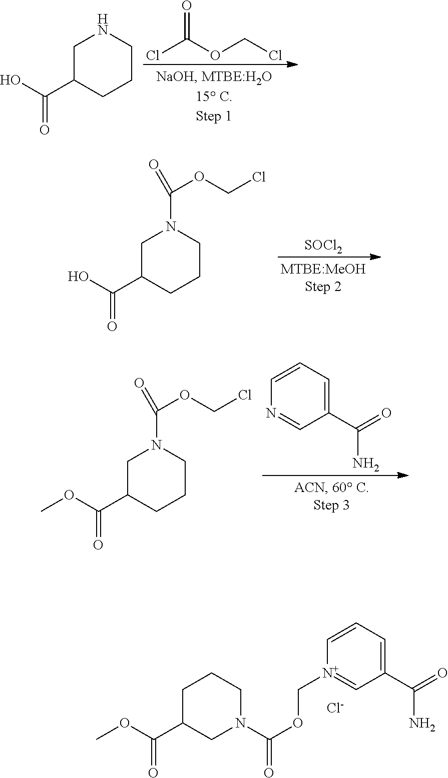

Step 1: Preparation of Amino Chloromethyl Carbamate with Piperidine-3-Carboxylic Acid

Piperidine-3-carboxylic acid (5.0 g, of 0.0387 mol., 1 eq.) was added to an oven dried 4 necked RBF at room temperature (25° C.). Methyl tert-butyl ether (MTBE) (25 mL, 5 vol) was added at 25° C. to the above RBF. The solution was stirred at 20-25° C. and then a solution of NaOH (3.0 g., 0.0774 mol., 2 eq.) in water (20 ml, 4 vol) was added dropwise through dropping funnel. The above reaction mixture was stirred at 20-25° C. for 20 min. The reaction mixture was cooled to 15° C. and chloromethyl chloroformate (CMCF) (9.98 g., 0.0774 mol., 2 eq.) was added dropwise. During the addition of CMCF, pH was maintained between 8.5 to 9.5 by addition of a solution of NaOH (4.6 gm, 0.1161 mol., 3 eq.) in water (10 mL, 2 vol) at 15-20°° C. After addition, reaction mixture was stirred for 1 hour at 15-20° C. (pH was maintained at 8.5-9.5 with addition of above NaOH solution). Reaction progress was monitored by HPLC. After consumption of starting material, MTBE (5 mL, 1 vol) was added and the reaction mixture was stirred for 20 min at 20-25° C. The aqueous layer was separated. Water (10 mL, 2 vol) was added to the organic layer and the mixtures was stirred for 20 min at 25° C. The aqueous layer was separated. The organic layer was concentrated under reduced pressure to obtain crude carbamate product (2 g.). Crude product was used in next step without further purification.

Step 2: Preparation of Methyl Ester of Chloromethyl Carbamate with Piperidine-3-Carboxylic Acid

Crude carbamate (2 g., 1.0 eq., 0.009 mol) was added at 25° C. under N₂ to a 4- necked RBF. MTBE (12 mL, 6 vol) and methanol (4 mL, 2 vol) were added at 25° C. under N2 to the above RBF. Thionyl chloride (1.30 mL, 2.0 eq., 0.018 mol.) was slowly added at 20-25° C. The reaction mixture was then stirred at RT for 14-16 hours. Reaction progress was monitored by HPLC. After consumption of starting material, reaction mixture was concentrated under reduced pressure at 40° C. The mixture was then cooled to 5-10° C. and purified water (8 mL, 4 vol) and MTBE (8 mL, 4 vol) were added. The reaction mixture was stirred for 2 hours. The aqueous layer was separated. Saturated sodium bicarbonate solution (8 mL, 4 vol) was added to the organic layer and the mixture was stirred for 2 hours at 10-15° C. The aqueous layer was separated and the organic layer was concentrated under reduced pressure to get methyl ester of carbamate (1.8 g.). Crude product was dried on rotavapor under vacuum and used in next step without further purification.

### Step 3: Preparation of Aminomethyl Carbamate Using Piperidine-3-Carboxylic Acid and Nicotinamide

Acetonitrile (14.5 mL, 8 vol) was added to crude methyl ester of carbamate product (1.8 g, 1.0 eq., 0.0061 mol.) at 25° C. under N₂ in a 4 necked dried RBF. Acetonitrile was subsequently distilled at 60° C. up to 4 vol. Reaction mixture was cooled to 35-40° C. Nicotinamide (0.74 g, 0.8 eq., 0.006 mol.) was added portion wise to above solution at 40-45° C. under N₂. The reaction mixture was heated at 60° C. and stirred for 22-24 hours. Reaction progress was monitored by HPLC. After consumption of starting material, reaction mixture was cooled to 25° C. n-Heptane (25 mL, 14 vol) and methyl isobutyl ketone (MIBK) (12.5 mL, 7 vol) were added to a separate 100 mL RBF and stirred at 20° C. for 10 min. Reaction mixture was added to the above RBF slowly at 20° C., resulting in formation of a slurry. The slurry was stirred at 20° C. for 5-6 hours. Suspension was filtered under N₂. The wet cake was washed by a cold solution of n-heptane (3.5 mL, 2 vol) and MIBK (2 mL, 1 vol) under N₂. Product was dried under vacuum. Purification by silica combi-flash column chromatography followed by recrystallization in dichloromethane and isopropyl acetate provided 2.1 gm of the product (15.2% overall yield for 3 steps). HPLC purity 98.9%.

### Characterization of Aminomethyl Carbamate Prepared Using Piperidine-3-Carboxylic Acid and Nicotinamide

The product exists as rotational isomers (rotamers) due to the restricted rotation around the carbamate functional group. Due to this, more ¹H and C¹³ NMR signals were seen than can be attributed to the structure. To demonstrate this fact, ¹H NMR was performed at 90° C. where the rotamers collapsed and provided the ¹H-NMR peaks that can be attributed to the structure (Table 5).

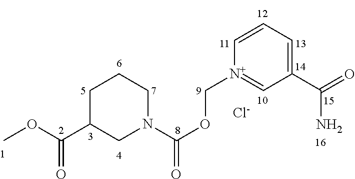 [SMILES: COC(=O)C1CCCN(C(=O)OC[n+]2cccc(C(N)=O)c2)C1 ; [Cl-]]

TABLE 5
Proton NMR at 90° C. in DMSO-d₆

| Proton # | δ (ppm) | Integration |
|---|---|---|
| 10 | 9.68 | 0.98, 1H |
| 13 | 9.28 | 0.99, 1H |
| 11 | 9.13 | 0.98, 1H |
| 16 NH₂ | 8.90-7.50, broad | 1.66, 2H |
| 12 | 8.30 | 1.11, 1H |
| 9 | 6.46 | 1.98, 2H |
| 4 | 3.88, 3.65 | 1.00, 1.02, 2H |
| 1 | 3.60 | 3.00, 3H |
| 7 | 3.30, 3.14 | 0.97, 1.19, 2H |
| 3 | 2.56 | 1.11, 1H |
| 5 | 1.92, 1.68 | 1.03, 1.05, 2H |
| 6 | 1.62, 1.45 | 1.05, 1.11, 2H |

C¹³ NMR (solvent CDC₁₃) was performed and there were more C¹³ peaks than can be attributed to the structure due to the presence of rotamers (Table 6).

TABLE 6
C¹³ NMR

| Carbon # | δ (ppm) |
|---|---|
| 2 | 173.22, 173.18 |
| 15 | 163.01, 162.96 |
| 8 | 152.96, 152.84 |
| 13 | 147.02, 146.94 |
| 10 | 146.71, 146.52 |
| 11 | 145.84 |
| 14 | 133.72 |
| 12 | 128.03, 127.91 |
| 9 | 81.20 |
| 1 | 52.03, 51.95 |
| 7 | 45.96, 45.76 |
| 4 | 44.84, 44.60 |
| 3 | 40.63, 40.42 |
| 5 | 26.73 |
| 6 | 24.38, 24.07 |

IR (film) vmax 3240, 3050, 2950, 2900, 1750, 1725,1700, 1420, 1380 cm⁻¹. UPLCMS m/z calculated for C₁₅H₂₀N₃O₅⁺ [M+H]⁺ 323.34, found 323.03.

Example 4—Preparation of Aminomethyl Carbamate Synthon Using Piperidine-2-Carboxylicacid and Isonicotinamide

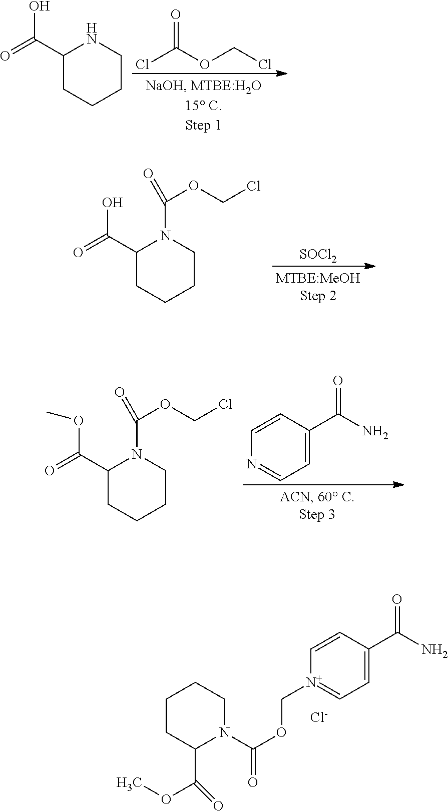

Step 1: Preparation of Chloromethyl Carbamate with Piperidine-2-Carboxylic Acid

Piperidine-2-carboxylic acid (5.0 g., 0.0387 mol., 1.0 eq.) was added to an oven dried 4-necked RBF at room temperature (25° C.). Methyl tert-butyl ether (MTBE) (25 mL, 5 vol) was added at 25° C. to the above RBF. The solution was stirred at 20-25° C. and a solution of NaOH (3.0 g., 0.0774 mol., 2 eq.) in water (20 mL, 4 vol) was added dropwise through a dropping funnel. The above reaction mixture stirred at 20-25° C. until clear solution (approximately 1 hour). Afterwards the reaction mixture was cooled to 15° C. Chloromethyl chloroformate (CMCF) (9.98 gm, 0.0774 mol., 2 eq.) was added dropwise. During addition of CMCF, pH was maintained between 8.5 to 9.5 by addition of a solution of NaOH (4.6 gm, 0.1161 mol., 3 eq.) in water (10 mL, 2 vol) at 15-20°° C. After addition was completed, reaction mixture was stirred for 1 hour at 15-20°° C. (pH was maintained at 8.5-9.5 with addition of the above NaOH solution). Reaction progress was monitored by HPLC. After consumption of starting material, MTBE (5 mL, 1 vol) was added and the reaction mixture was stirred for 20 min at 20-25° C. The aqueous layer was separated. Water (10 mL, 2 vol) was added to the organic layer and the reaction mixture was stirred for 20 min at 25° C. The aqueous layer was separated. The organic layer was concentrated under reduced pressure to obtain crude carbamate (3.6 g.). Crude product was used in next step without further purification.

### Step 2: Preparation of Methyl Ester of Chloromethyl Carbamate of Piperidine-2-Carboxylic Acid

Crude carbamate (3.5 g., 1.0 eq., 0.015 mol.) was added at 25° C. under N₂ to a 4-necked RBF. MTBE (21 mL, 6 vol) and methanol (7 mL, 2 vol) were added at 25° C. under N₂ to the above RBF. Thionyl chloride (2.29 mL, 2.0 eq., 0.031 mol.) was slowly added at 20-25° C. The reaction was then stirred at room temperature for 14-16 hours. Reaction progress was monitored by HPLC. After consumption of starting material, reaction mixture was concentrated under reduced pressure at 40° C. The flask was cooled to 5-10° C. and purified water (14 mL, 4 vol) and MTBE (14 mL, 4 vol) were added. The reaction mixture was stirred for 2 hours. The aqueous layer was separated. Saturated sodium bicarbonate solution (14 mL, 4 vol) was added to the organic layer and the mixture was stirred for 2 hours at 10-15° C. The aqueous layer was separated. The organic layer was concentrated under reduced pressure to get methyl ester of carbamate (3 g.). Crude product was used in next step without further purification.

### Step 3: Preparation of Aminomethyl Carbamate Using Piperidine-2-Carboxylicacid and Isonicotinamide

Acetonitrile (60 mL, 8.0 vol) was added to the crude methyl ester of carbamate (3 g, 1.0 eq., 0.012 mol.) at 25° C. under N₂ in a 4 necked dried RBF. Acetonitrile was subsequently distilled at 60° C. up to 4 vol. Reaction mixture cooled to 35-40° C. Isonicotinamide (1.24 g, 0.8 eq., 0.010 mol.) was added portion wise to the above solution at 40-45° C. under N₂. The reaction mixture was heated at 60° C. and stirred for 22-24 hours. Reaction progress was monitored by HPLC. After consumption of starting material, reaction mixture was cooled to 25° C. n-Heptane (42 mL, 14 vol) and methyl isobutyl ketone (MIBK) (21 mL, 7 vol) were added to a separate 100 mL RBF and stirred at 20° C. for 10 min. Reaction mixture was added to the above RBF slowly at 20° C., resulting in the formation of a slurry. The slurry was stirred at 20° C. for 5-6 hours. Suspension was filtered under N₂. The wet cake was washed with a cold mixture of n-heptane (7 mL, 2 vol) and MIBK (3 mL, 1 vol) under N₂ and dried under vacuum. Purification of crude product by silica combi-flash column chromatography followed by recrystallization in dichloromethane and isopropyl acetate gave product (5.8 g, overall yield 42.2% for three steps). HPLC purity=95.3%.

### Characterization of Aminomethyl Carbamate Prepared Using Piperidine-2-Carboxylic Acid and Isonicotinamide

The product exists as rotational isomers (rotamers) due to the restricted rotation around the carbamate functional group. Due to this, more ¹H and C¹³ NMR signals were seen than can be attributed to the structure. Proton and C₁₃ NMR details are provided in Tables 7 and 8, respectively.

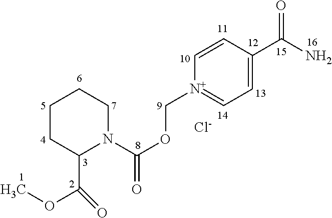 [SMILES: COC(=O)C1CCCCN1C(=O)OC[n+]1ccc(C(N)=O)cc1 ; [Cl-]]

TABLE 7
Proton NMR in CDCl₃

| Proton # | δ (ppm) |
|---|---|
| 10, 14 | 9.78, 9.50, 2H |
| 11, 13 | 8.96, 2H |
| 16-NH₂ | 6.82, 2H |
| 9 | 6.64, 2H |
| 3 | 4.85, 1H |
| 7 | 4.00, 3.00, 2H |
| 1 | 3.82, 3H |
| 4 | 2.95, 2.25, 2H |
| 5, 6 | 1.22-1.50, 2H |

C¹³ NMR of sample in CDCl₃ was performed and there were more peaks than accounted for the structure due to the presence of rotamers.

TABLE 8
C¹³ NMR

| Carbon # | δ (ppm) |
|---|---|
| 2 | 171.15, 171.23 |
| 15 | 163.48 |
| 8 | 154.38, 153.83 |
| 12 | 150.19, 150.10 |
| 10 | 145.98 |
| 14 | 145.88 |
| 10 | 133.72 |
| 11 | 126.97 |
| 13 | 126.83 |
| 9 | 80.99, 80.78 |
| 3 | 55.14, 54.88 |
| 1 | 52.88, 52.59 |
| 7 | 42.54 |
| 4 | 26.40 |
| 6 | 24.35, 24.13 |
| 5 | 20.37 |

IR (film) vmax 3360, 3150, 3050, 2950, 1760, 1730, 1700, 1450, 1400 cm⁻¹. UPLCMS m/z calculated for C₁₅H20N305+ [M+H]⁺ is 323.34, found 323.25.

### Example 5—Synthesis of Compound V

The synthetic strategy of forming aminomethyl carbamate synthon in presence of carboxylic was further studied.

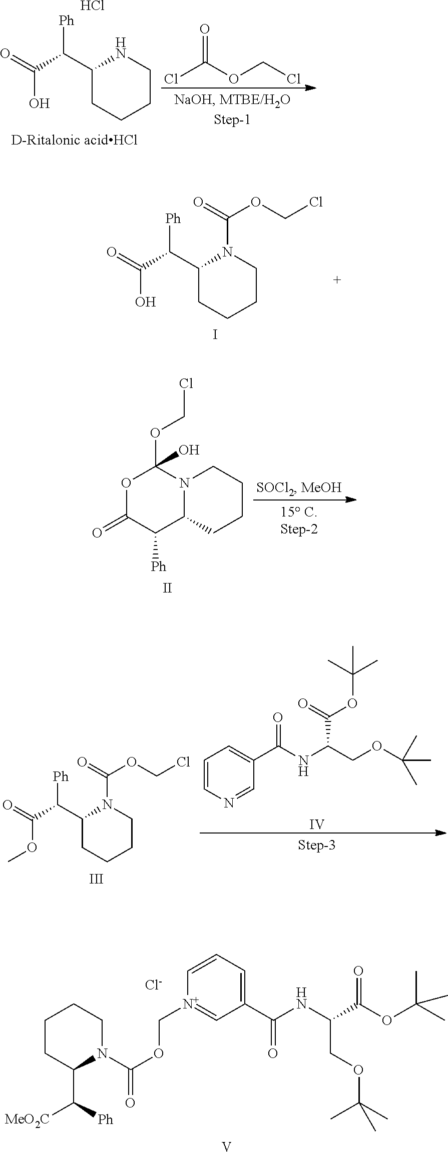

This process involved 3 steps and did not involve the formation of dexmethylphenidate (DMP). This process starts with D-ritalonic acid hydrochloride which on treatment with chloromethyl chloroformate results in a mixture of compounds I and II (Step 1). Compound II is formed due to internal cyclization of compound I. Both Compounds I and II convert to Compound III on treatment with thionyl chloride in methanol or methanol/MTBE (Step 2). Coupling of the Compound III with Compound IV provides Compound V (Step 3). The overall yield of three process steps is about 68%. Deprotection of t-butyl groups provides a deprotected compound.

### Step 1: Preparation of Compounds I and II

D-Ritalonic acid hydrochloride (16 g, 62 mmol, 1.0 eq.) was added to a 4 necked RBF. Methyl tert-butyl ether (MTBE) (80 mL, 5 vol) was added at 25° C. to the above RBF. The resulting mixture was stirred at 10-15° C. and a solution of NaOH (5 g, 125 mmol, 2 eq.) in water (64 ml) was added dropwise through a dropping funnel. The mixture was stirred at 10-15° C. for 30 min. Afterwards, chloromethyl chloroformate (CMCF) (16.1 g, 125 mmol, 2 eq.) was slowly added. During the addition of CMCF, pH was maintained between 8.5 to 9.5 by adding a solution of NaOH (7.5 g, 180 mmol, 3 eq) in water (24 mL) as required at 10-15° C. After addition was completed, the reaction mixture was stirred for 2-3 hours at 10-15° C. (pH was maintained at 8.5-9.5 with addition of the above NaOH solution). Reaction progress was monitored by HPLC. When the starting D-Ritalonic acid was below 3%, the reaction mixture was neutralized to a pH of 6.5 to 7 by addition of conc. HCl (approximately 5 mL) at 10-15° C. MTBE (48 mL, 3 vol) was then added and the reaction mixture was stirred for 30 min at 25° C. The aqueous layer was separated. The organic layer was concentrated under reduced pressure to obtain a mixture of compounds I and II as an oil (21 g). Crude product was used in step 2 without further purification.

### Step 2: Preparation of Compound III

Crude product from step 1 (21 g, obtained from 16 g of D-Ritalonic acid HCl in step 1) was added to a 4-necked RBF at 25° C. under N₂. Methanol (80 mL, 5 vol) was added to the RBF at 25° C. under N₂. Thionyl chloride (9.3 ml, 2.0 eq.) was slowly added while maintaining internal temperature at 10-15° C. The reaction was then stirred at 10-15° C. and aged for 16 hours (gradual haziness occurred during the stirring). Reaction progress was monitored by HPLC. After completion, the reaction mixture was concentrated under reduced pressure. The flask was cooled to 5-10° C. and deionized water (64 mL, 4 vol) and MTBE (80 mL, 5 vol) were added. The reaction mixture was stirred for 1 hour. The aqueous layer was separated. A saturated sodium bicarbonate solution (48 mL, 3 vol) was added to the organic layer and the mixture was stirred for 1 hour at 10-15° C. The aqueous layer was separated. The organic layer was concentrated under reduced pressure to get crude Compound III as an oil (18.5 g). Crude product was dried under vacuum (using rotavapor) and used in next step without further purification. HPLC purity 91.21%.

### Step 3: Preparation of Compound V

Acetonitrile (78 mL, 4.0 vol) was added to the crude Compound III (18.5 g, 1.0 eq., 56 mmol.) at 25° C. under N₂. Acetonitrile was subsequently distilled at 100° C. up to 1.5 vol. Reaction mixture was cooled to 40-45° C. Solution of Compound IV (14.6 g, 0.8 eq., 45 mmol) in acetonitrile (18.5 mL, 1.0 vol) was added slowly to the above solution at 40-45° C. under N₂. The reaction mixture was heated at 60° C. and stirred for 35-40 hours. Reaction progress was monitored by HPLC. After completion of the reaction, the mixture was cooled to 20° C. n-Heptane (259 mL, 14 vol) and methyl isobutyl ketone (MIBK) (111 mL, 6 vol) were added to a separate RBF (1 L), followed by Compound V seed crystal (0.04 g). The contents were stirred at 20° C. for 30 min. The Compound V reaction mixture was added to the RBF (1L) over 1 hour at 20° C., resulting in the formation of a slurry. The slurry was stirred at 20° C. for 16 hours. Suspension was filtered at 20° C. under N₂. The wet cake was washed with a cold mixture of n-heptane (74 mL, 4 vol) and MIBK (27 mL, 1.5 vol). The solid was dried under vacuum at 45° C. for 12 hours to afford Compound V (27.5 g). Three step overall yield about 68% from D-Ritalonic acid hydrochloride. HPLC purity 97.35%.

### Characterization of Compound V

The product exists as rotational isomers (rotamers) due to the restricted rotation around the carbamate functional group. Due to this, more ¹H and C¹³ NMR signals were seen than can be accounted for the structure. Proton and C¹³ NMR details are provided in Tables 9 and 10, respectively.

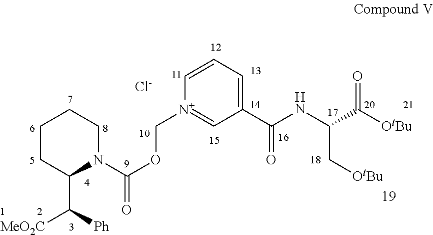 [SMILES: COC(=O)[C@H](c1ccccc1)[C@H]1CCCCN1C(=O)OC[n+]1cccc(C(=O)N[C@@H](COC(C)(C)C)C(=O)OC(C)(C)C)c1 ; [Cl-]]

TABLE 9
¹H NMR in MeOD

| Proton # | δ (ppm) | Integration |
|---|---|---|
| 15 | 9.74, 9.64 | 1 H |
| 11 | 9.44, 9.38 | 1 H |
| 13 | 9.10 | 1H |
| 12 | 8.34 | 1 H |
| 3-Ph | 7.50-7.26 | 5 H |
| 10 | 6.80, 6.62 | 1 H |
| 10 | 6.45 | 1 H |
| 17 | 4.96, 4.84 | 1H |
| NH | 4.74 | 1H |
| 3 | 4.32, 4.26 | 1H |
| 4 | 4.14, 4.02 | 1H |
| 18 | 3.90, 3.72 | 2H |
| 1 | 3.30 | 3H |
| 8 | 3.40, 3.25, 3.10 | 2H |
| 5, 6, 7 | 1.80-1.45 | 6H |
| 21-^{t}Bu | 1.50, 1.40 | 9H |
| 19-^{t}Bu | 1.20, 1.10 | 9H |

TABLE 10
C¹³ NMR in MeOD

| Carbon # | δ (ppm) |
|---|---|
| 20 | 173.05 |
| 2 | 172.43 |
| 16 | 168.90 |
| 9 | 162.17, 162.07 |
| 11, 12, 13, | 153.02, 152.78, 147.14, 146.98, 145.99, |
| 14, 15 | 145.08, 144.59 |
| Phenyl | 135.56, 135.41, 134.51, 134.44, 129.05, |
| 6 carbons | 128.75, 128.57, 128.50, 128.29, 128.19, |
| | 127.87, 127.74, 127.63 |
| 10 | 82.03, 80.97 |
| 19, 21 | 73.35, 73.28 |
| 1, 4, 17, 18 | 61.40, 57.85, 54.86, 54.65, 54.49, 53.94, |
| | 52.03, 51.44, 51.24, 50.27, 50.12 |
| 3, 8 | 40.15, 39.63 |
| 19-Butyl | 26.93, 26.31, 26.24 |
| 21-Butyl | |
| 5, 6, 7 | 24.94, 24.74, 24.64 |

UPLCMS m/z calculated for C₃₃H₄₆N₃O₈⁺ [M+H]⁺ is 613.74, found 613.56.

The present disclosure describes a process to prepare aminomethyl carbamate synthon in presence of carboxylic acid in three steps. To demonstrate this synthesis, four analogues were prepared using different piperidine carboxylic acids and different nicotinamides. Finally, this strategy was applied for preparation of a Compound V.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A process for preparation of a compound of Formula (I): 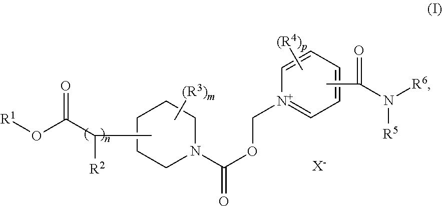
wherein
R¹ is C₁-C₆ alkyl;
R² is independently selected from the group consisting of H, C₁-C₆ alkyl, C₃-C₇ cycloalkyl, C₄-C₁₃ cycloalkylalkyl, heterocyclyl, aryl, and heteroaryl, wherein C₁-C₆ alkyl, C₃-C₇ cycloalkyl, C₄-C₁₃ cycloalkylalkyl, heterocyclyl, aryl, and heteroaryl can be optionally substituted from 1 to 3 times with a substituent selected independently at each occurrence from the group consisting of C₁-C₆ alkyl, OH, halogen, and C₁-C₆ alkoxy;
R³ is independently selected at each occurrence thereof from the group consisting of H, C₁-C₆ alkyl, C₃-C₇ cycloalkyl, C₄-C₁₃ cycloalkylalkyl, heterocyclyl, aryl, and heteroaryl;
R⁴ is independently selected at each occurrence thereof from the group consisting of H, C₁-C₆ alkyl, C₃-C₇ cycloalkyl, C₄-C₁₃ cycloalkylalkyl, heterocyclyl, aryl, and heteroaryl;
R⁵ is selected from the group consisting of H, C₁-C₆ alkyl, C₃-C₇ cycloalkyl, C₄-C₁₃ cycloalkylalkyl, heterocyclyl, aryl, and heteroaryl, wherein C₁-C₆ alkyl, C₃-C₇ cycloalkyl, C₄-C₁₃ cycloalkylalkyl, heterocyclyl, aryl, and heteroaryl can be optionally substituted from 1 to 3 times with a substituent selected independently at each occurrence from the group consisting of —OC₁-C₆ alkyl, —C(O)OH, and —C(O)OC₁-C₆ alkyl;
R⁶ is selected from the group consisting of H, C₁-C₆ alkyl, C₃-C₇ cycloalkyl, C₄-C₁₃ cycloalkylalkyl, heterocyclyl, aryl, and heteroaryl, wherein C₁-C₆ alkyl, C₃-C₇ cycloalkyl, C₄-C₁₃ cycloalkylalkyl, heterocyclyl, aryl, and heteroaryl can be optionally substituted from 1 to 3 times with a substituent selected independently at each occurrence from the group consisting of —OC₁-C₆ alkyl, —C(O)OH, and —C(O)OC₁-C₆ alkyl;
X is halogen, mesylate, tosylate, or triflate;
n is 0, 1, 2, 3, 4, 5, or 6;
m is 0, 1, 2, 3, 4, 5, 6, 7, 8, or 9; and
p is 0, 1, 2, 3, or 4;
or a solvate thereof,
said process comprising:
providing an intermediate compound of Formula (IVa) and/or Formula (IVb): 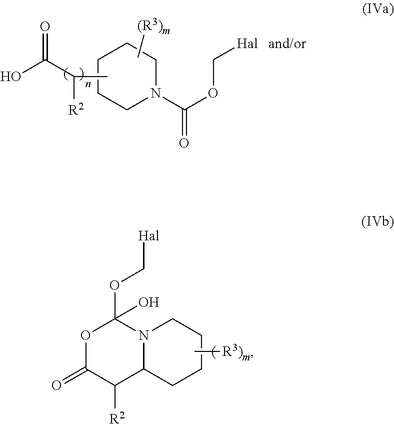
wherein Hal is halogen;
reacting the intermediate compound of Formula (IVa) and/or Formula (IVb) with an alkylating agent to produce an intermediate compound of Formula (II) having the structure: 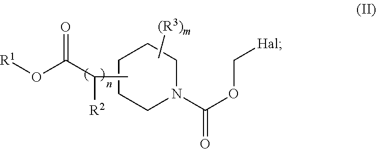
and
reacting the intermediate compound of Formula (II) with a compound of Formula (III): 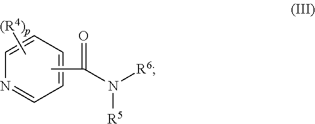
to produce the compound of Formula (I).

2. The process according to claim 1, wherein n is 0 or 1.

3. (canceled)

4. The process according to claim 1, wherein R¹ is methyl.

5. The process according to claim 1, wherein R⁵ is H.

6. The process according to claim 1, wherein the compound of Formula I has the structure selected from the group consisting of

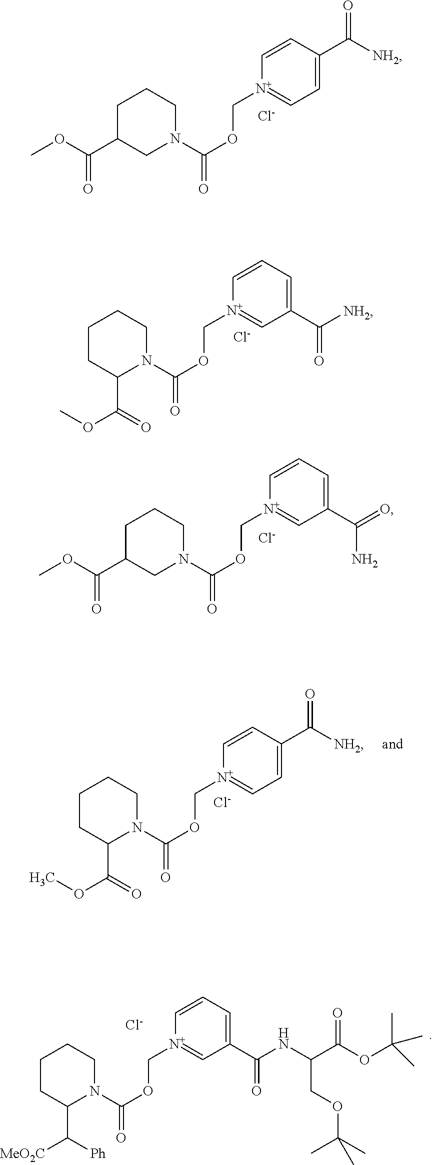

7.-8. (canceled)

9. The process according to claim 1, wherein the compound of Formula (II) has the structure selected from the group consisting of:

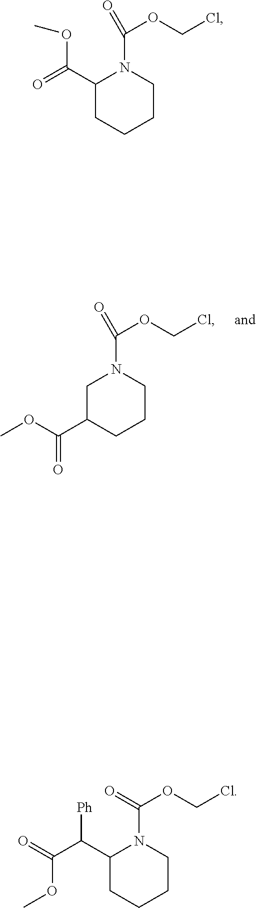 [SMILES: COC(=O)C1CCCCN1C(=O)OCCl ; COC(=O)C1CCCN(C(=O)OCCl)C1 ; COC(=O)C(c1ccccc1)C1CCCCN1C(=O)OCCl]

10. (canceled)

11. The process according to claim 1, wherein the compound of Formula (III) has the structure selected from the group consisting of:

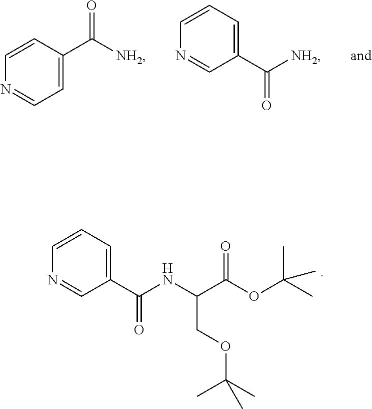 [SMILES: NC(=O)c1ccncc1 ; NC(=O)c1cccnc1 ; "and" ; CC(C)(C)OCC(NC(=O)c1cccnc1)C(=O)OC(C)(C)C]

12. (canceled)

13. The process according to claim 1, wherein said reacting the intermediate compound of Formula (II) with the compound of Formula (III) is carried out at a temperature of from about 25° C. to about 80° C.

14. (canceled)

15. The process according to claim 1, wherein said forming the intermediate compound of Formula (II) comprises:
(i) reacting the intermediate compound of Formula (IVa) with an alkylating agent to produce the intermediate compound of Formula (II); or
(ii) reacting the intermediate compound of Formula (IVb) with an alkylating agent to produce the intermediate compound of Formula (II); or
(iii) reacting a mixture of the intermediate compounds of Formula (IVa) and Formula (IVb) with an alkylating agent to produce the intermediate compound of Formula (II).

16.-17. (canceled)

18. The process according to claim 1, wherein the alkylating agent is SOCl₂/C₁₋₆ alkyl-OH.

19. The process according to claim 1, wherein the intermediate compound of Formula (IVa) has the structure selected from the group consisting of:

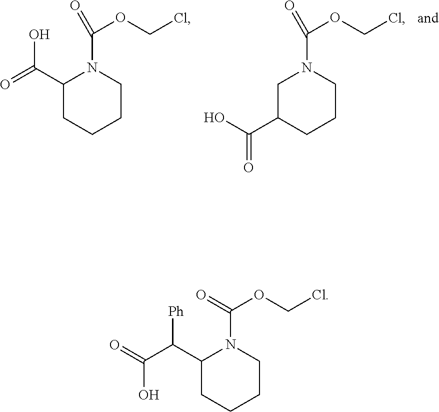 [SMILES: O=C(O)C1CCCCN1C(=O)OCCl ; O=C(O)C1CCCN(C(=O)OCCl)C1 ; O=C(O)C(c1ccccc1)C1CCCCN1C(=O)OCCl]

20. (canceled)

21. The process according to claim 1, wherein the intermediate compound of Formula (IVb) is

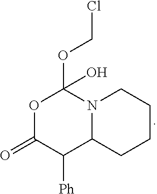 [SMILES: O=C1OC(O)(OCCl)N2CCCCC2C1c1ccccc1]

22. (canceled)

23. The process according to claim 1 further comprising:
providing a compound of Formula (V): 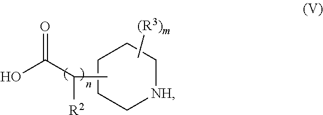
and
forming the intermediate compound of Formula (IVa) and/or (IVb) from the compound of Formula (V).

24. The process according to claim 23, wherein said forming the intermediate compound of Formula (IVa) and/or (IVb) comprises:
reacting the compound of Formula (V) with a compound of Formula (VI): 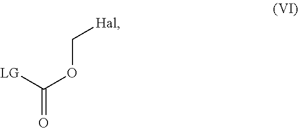
wherein LG is a suitable leaving group.

25. The process according to claim 24, wherein the compound of Formula (VI) is chloromethyl chloroformate.

26. The process according to claim 24, wherein said reacting the compound of Formula (V) with the compound of Formula (VI) is carried out in the presence of a base.

27. (canceled)

28. The process according to claim 24, wherein said reacting the compound of Formula (V) with the compound of Formula (VI) is carried out at a temperature of from about 5° C. to about 25° C.

29.-30. (canceled)

31. The process according to claim 23, wherein the compound of Formula (V) is selected from the group consisting of: 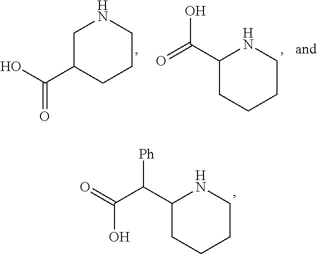
or a salt thereof.

32. (canceled)

33. A compound of Formula (I): 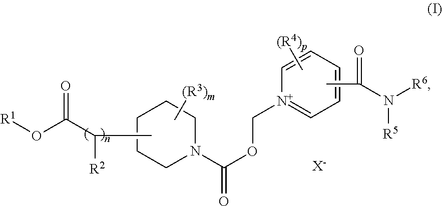
wherein
R¹ is C₁-C₆ alkyl;
R² is independently selected from the group consisting of H, C₁-C₆ alkyl, C₃-C₇ cycloalkyl, C₄-C₁₃ cycloalkylalkyl, heterocyclyl, aryl, and heteroaryl, wherein C₁-C₆ alkyl, C₃-C₇ cycloalkyl, C₄-C₁₃ cycloalkylalkyl, heterocyclyl, aryl, and heteroaryl can be optionally substituted from 1 to 3 times with a substituent selected independently at each occurrence from the group consisting of C₁-C₆ alkyl, OH, halogen, and C₁-C₆ alkoxy;
R³ is independently selected at each occurrence thereof from the group consisting of H, C₁-C₆ alkyl, C₃-C₇ cycloalkyl, C₄-C₁₃ cycloalkylalkyl, heterocyclyl, aryl, and heteroaryl;
R⁴ is independently selected at each occurrence thereof from the group consisting of H, C₁-C₆ alkyl, C₃-C₇ cycloalkyl, C₄-C₁₃ cycloalkylalkyl, heterocyclyl, aryl, and heteroaryl;
R⁵ is selected from the group consisting of H, C₁-C₆ alkyl, C₃-C₇ cycloalkyl, C₄-C₁₃ cycloalkylalkyl, heterocyclyl, aryl, and heteroaryl, wherein C₁-C₆ alkyl, C₃-C₇ cycloalkyl, C₄-C₁₃ cycloalkylalkyl, heterocyclyl, aryl, and heteroaryl can be optionally substituted from 1 to 3 times with a substituent selected independently at each occurrence from the group consisting of —OC₁-C₆ alkyl, —C(O)OH, and —C(O)OC₁-C₆ alkyl;
R⁶ is selected from the group consisting of H, C₁-C₆ alkyl, C₃-C₇ cycloalkyl, C₄-C₁₃ cycloalkylalkyl, heterocyclyl, aryl, and heteroaryl, wherein C₁-C₆ alkyl, C₃-C₇ cycloalkyl, C₄-C₁₃ cycloalkylalkyl, heterocyclyl, aryl, and heteroaryl can be optionally substituted from 1 to 3 times with a substituent selected independently at each occurrence from the group consisting of —OC₁-C₆ alkyl, —C(O)OH, and —C(O)OC₁-C₆ alkyl;
X is halogen, mesylate, tosylate, or triflate;
n is 0, 1, 2, 3, 4, 5, or 6;
m is 0, 1, 2, 3, 4, 5, 6, 7, 8, or 9; and
p is 0, 1, 2, 3, or 4;
or a solvate thereof,
with the proviso that when n is 1; R¹ is Me; m is 0; and p is 0, R² is not Ph.

34. A compound of Formula (II): 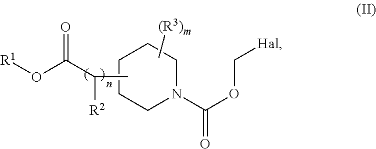
and
wherein
Hal is halogen;
R¹ is H or C₁-C₆ alkyl;
R² is independently selected from the group consisting of H, C₁-C₆ alkyl, C₃-C₇ cycloalkyl, C₄-C₁₃ cycloalkylalkyl, heterocyclyl, aryl, and heteroaryl, wherein C₁-C₆ alkyl, C₃-C₇ cycloalkyl, C₄-C₁₃ cycloalkylalkyl, heterocyclyl, aryl, and heteroaryl can be optionally substituted from 1 to 3 times with a substituent selected independently at each occurrence from the group consisting of C₁-C₆ alkyl, OH, halogen, and C₁-C₆ alkoxy;
R³ is independently selected at each occurrence thereof from the group consisting of H, C₁-C₆ alkyl, C₃-C₇ cycloalkyl, C₄-C₁₃ cycloalkylalkyl, heterocyclyl, aryl, and heteroaryl;
n is 0, 1, 2, 3, 4, 5, or 6; and
m is 0, 1, 2, 3, 4, 5, 6, 7, 8, or 9;
or a pharmaceutically acceptable salt thereof or a solvate thereof,
with the proviso that when n is 1; R¹ is Me; m is 0; and p is 0, R² is not Ph.

35.-36. (canceled)
